(19) United States
(12) Patent Application Publication
(10) Pub. No.: US 2026/0224324 A1
(43) Pub. Date: Aug. 6, 2026

(54) CANNULA ASSEMBLY FOR COMMUNICATING WITH AN AUGMENTED REALITY SYSTEM

(71) Applicant: New View Medical, Inc., Newton Center, MA (US)

(72) Inventors: Bryce C. Klontz, JR., Boston, MA (US); Joseph Peter Corrigan, Hardwick, Cambridgeshire (GB); Rachel Mary Rakvica, Newbury, MA (US)

(73) Assignee: New View Medical, Inc., Newton Center, MA (US)

(21) Appl. No.: 19/157,424

(22) PCT Filed: Mar. 7, 2024
(86) PCT No.: PCT/US2024/018789

§ 371 Date: Aug. 18, 2025

Related U.S. Application Data
(60) Provisional application No. 63/489,485, filed on Mar. 10, 2023.

(51) Int. Cl. A61B 90/00; A61B 1/00; A61B 1/05; A61B 1/313; A61B 17/00; A61B 17/34; G06F 3/01; G06T 19/00; G10L 15/22; H04N 23/50; H04N 23/51

(52) U.S. Cl. CPC A61B 90/37; A61B 1/00048; A61B 1/05; A61B 1/3132; A61B 17/3421; G06F 3/014; G06F 3/017; G06T 19/006; G10L 15/22; H04N 23/51; H04N 23/555; A61B 2017/00207; A61B 2090/365; A61B 2090/372; G06T 2210/41; G10L 2015/223

(57) ABSTRACT

A surgical system is disclosed for performing a surgical procedure on a patient. The system may include a cannula assembly having a cannula tube, the cannula tube having one or more housings coupled thereto. The housings are positioned within the patient when the distal end of the cannula tube is inserted into the patient, and are movable relative to the cannula tube between closed and open positions. The housings may each include an image sensor configured to provide image data of the patient's anatomy when the housings are in the open position within the patient. The system may also include an augmented reality display device, e.g., augmented reality goggles or headset, configured to receive the image data and to display the image data to a user. The system may also include a microphone and/or gesture control input device, via which the processor may receive voice and/or gesture control signals so as to control at least one attribute of the image displayed to the user via the augmented reality display device.

### CROSS-REFERENCE TO RELATED APPLICATIONS

This application claims the benefit of U.S. Provisional Application Ser. No. 63/489,485, filed Mar. 10, 2023, the complete disclosure of which is incorporated herein by reference for all purposes.

### BACKGROUND

Minimally invasive surgery involves making small incisions into a body of a patient to insert surgical tools. For example, a surgeon may perform a laparoscopic procedure using multiple cannulas inserted through individual incisions that accommodate various surgical tools, including illumination devices and imaging devices. To accomplish the insertion, cannula assemblies may be used to puncture the body cavity. A cannula assembly often includes an obturator and a cannula. An obturator is a guide placed inside a cannula, the obturator having either a sharp tip (e.g., a pointed cutting blade) or a blunt tip for creating an incision or opening in the patient for the cannula to pass through. After the obturator and cannula are inserted, the obturator is removed, leaving the cannula in place for use in inserting the surgical tools into the surgical space within a patient. Typically, in addition to cannulas forming individual incisions for surgical tools, an individual incision may also be made through the patient by a cannula that is thereafter dedicated to holding an illumination and/or imaging device, e.g., a traditional endoscope or laparoscope. A surgical tool combining a cannula and an imaging device in a single unit is disclosed, for example, in U.S. Pat. No. 8,834,358, the disclosure of which is herein incorporated by reference in its entirety.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

Described hereinbelow, in accordance with various embodiments thereof, is a surgical system for performing a surgical procedure on a patient. The system may include a cannula assembly including a cannula tube having a longitudinal axis, a proximal end portion and a distal end portion configured for insertion into a patient. The cannula tube may have a housing coupled to the cannula tube between the proximal and distal ends of the cannula tube so as to be positioned within the patient when the distal end of the cannula tube is inserted into the patient. The housing may be movable relative to the cannula tube between a closed position and an open position. In addition, the housing may include an image sensor configured to provide image data of the patient's anatomy when the housing is in the open position within the patient. The system may also include an augmented reality display device configured to receive the image data and to display the image data to a user.

In various embodiments, the augmented reality display device may include a pair of augmented reality goggles configured to be worn by the user. Still further, the system may also include an augmented reality processor configured to process the image data for display to the user via the augmented reality display device. In some embodiments, the system may also include a microphone, and the augmented reality processor may be configured to receive a voice control signal from the microphone. In such an embodiment, the processor may also be configured to process the voice control signal so as to control at least one attribute, e.g., its position, its zoom, its brightness, etc., of the image displayed to the user via the augmented reality display device. The microphone may, in embodiments, be mounted directly on the augmented reality display device.

In still further embodiments, the surgical system may also include a gesture control input device. The augmented reality processor may be configured to receive a gesture control signal from the gesture control input device. Still further, the processor may be configured to process the gesture control signal to control at least one attribute of the image displayed to the user via the augmented reality display device. In embodiments, the gesture control input device may include one of a glove or a fingertip sensor.

In still further embodiments, the surgical system may include a second imaging device configured for insertion into a patient. The second imaging device may provide second image data of the patient's anatomy. Also included may be an image processor configured to receive the first and second image data and to process the first and second image data so as to generate a combined image, e.g., a stereoscopic or 3D image, displayed to the user via the augmented reality display device. In some embodiments, the cannula assembly may include a second housing coupled to the cannula tube between the proximal and distal ends of the cannula tube so as to be positioned within the patient when the distal end of the cannula tube is inserted into the patient. The second housing may be movable relative to the cannula tube between a closed position and an open position. The second imaging device may be an image sensor mounted in the second housing. The cannula assembly may also include spatial data components that provide spatial data related to the position of the cannula assembly, and the image processor may be configured to generate the combined image based at least in part on the spatial data.

In still further embodiments, there is provided a surgical system for performing a surgical procedure on a patient, the system including a cannula assembly. The cannula assembly may include a cannula tube having a longitudinal axis, a proximal end portion and a distal end portion configured for insertion into a patient. The cannula tube may have a housing coupled to the cannula tube between the proximal and distal ends of the cannula tube so as to be positioned within the patient when the distal end of the cannula tube is inserted into the patient. The housing may be movable relative to the cannula tube between a closed position and an open position. The housing may include an image sensor configured to provide image data of the patient's anatomy when the housing is in the open position within the patient. The system may also include a pair of augmented reality display goggles configured to be worn by a user and to display the image data to a user.

In various embodiments, the surgical system may also include an augmented reality processor configured to process the image data for display via the pair of augmented reality display goggles. A microphone may be mounted on the augmented reality display goggles, and the augmented reality processor may be configured to receive a voice control signal from the microphone. The augmented reality processor may also be configured to process the voice control signal so as to control at least one attribute of the image displayed to the user via the augmented reality display goggles. Advantageously, the microphone may be mounted directly on the augmented reality display device.

In still further embodiments, the surgical system may also include a gesture control input device. The augmented reality processor may be configured to receive a gesture control signal from the gesture control input device. The augmented reality processor may also be configured to process the gesture control signal to control at least one attribute of the image displayed to the user via the augmented reality display goggles. The gesture control input device may include one of a glove or a fingertip sensor.

The surgical system may also include a second imaging device configured for insertion into a patient and to provide second image data of the patient's anatomy. An image processor may be configured to receive the first and second image data and to process the first and second image data so as to generate a combined image displayed to the user via the augmented reality display goggles. In embodiments, the cannula assembly may include a second housing coupled to the cannula tube between the proximal and distal ends of the cannula tube so as to be positioned within the patient when the distal end of the cannula tube is inserted into the patient. The second housing may be movable relative to the cannula tube between a closed position and an open position. The second imaging device may be an image sensor mounted in the second housing. Still further, the cannula assembly may include spatial data components that provide spatial data related to the position of the cannula assembly. The image processor may be configured to generate the combined image based at least in part on the spatial data.

Also provided is a surgical system, for performing a surgical procedure on a patient, that includes a cannula assembly including a cannula tube having a longitudinal axis, a proximal end portion and a distal end portion configured for insertion into a patient. The cannula tube may have first and second housings coupled to the cannula tube between the proximal and distal ends of the cannula tube so as to be positioned within the patient when the distal end of the cannula tube is inserted into the patient. The housings may be movable relative to the cannula tube between a closed position and an open position. The housings may include first and second image sensors, respectively. The first and second image sensors may be configured to provide first and second image data, respectively, of the patient's anatomy when the housings are in the open position within the patient. The system may also include an image processor that is configured to receive the first and second image data and to process the first and second image data so as to generate a combined image. Still further, the system may include an augmented reality display device configured to receive the image data and to display the image data to a user. The augmented reality display device may be a pair of augmented reality display goggles configured to be worn by a user.

The system may also include, in embodiments, a microphone. The image processor may be configured to receive a voice control signal from the microphone and to process the voice control signal so as to control at least one attribute of the image displayed to the user via the augmented reality display device. Additionally or alternatively, the system may include a gesture control input device. The image processor may be configured to receive a gesture control signal from the gesture control input device and to process the gesture control signal to control at least one attribute of the image displayed to the user via the augmented reality display device.

Among various other advantages provided by certain embodiments as will be evident from the Detailed Description below, there may also be the benefit that fewer punctures through a patient, e.g., through an abdominal wall or other bodily surface, are made during a surgical procedure. As set forth above, a surgeon typically performs a laparoscopic procedure using multiple cannulas inserted through individual incisions, wherein at least one such cannula and incision is occupied by an illumination/imaging device, such as a traditional endoscope and/or laparoscope. According to various embodiments thereof, there is provided a cannula assembly and/or system therefor that eliminates the need for this separate puncture by a cannula assembly for an endoscope/laparoscope, since it provides, in certain embodiments, a cannula assembly which provides both an illumination/imaging device (e.g., mounted or coupled to the cannula tube) and an internal lumen through which a separate surgical tool (e.g., a surgical stapler, etc.) may be inserted. The reduction of at least puncture during a surgical procedure, as may be enabled in certain embodiments, may improve the safety of the surgical procedure by avoiding potential complications, reducing pain and/or speeding the patient's recovery.

### BRIEF DESCRIPTION OF THE DRAWINGS

FIG. 1 shows a cannula assembly, in accordance with various embodiments.

FIG. 2 shows a system including two cannula assemblies, in accordance with various embodiments.

FIG. 3 is a system block diagram that illustrates two cannula assemblies employed in a surgical procedure, in accordance with various embodiments.

FIG. 4 shows a block diagram illustrating an example of a device controller in accordance with various embodiments.

FIG. 5 shows a block diagram illustrating an example of an imaging controller for a system in accordance with aspects.

### DETAILED DESCRIPTION

Generally, described hereinbelow are imaging systems and, more particularly, endoscopic imaging systems. Systems and methods in accordance with various embodiments provide a cannula assembly that includes two or more imaging sensors configured to provide image data related to a surgical site. In some embodiments, the image data that is provided by the cannula assembly's first imaging sensor is combined with image data from the cannula assembly's second imaging sensor into a combined image that is displayed to a user via an augmented reality headset.

In various embodiments, the combination of those image streams can employ relative spatial information of the cannula assemblies to enable the image data streams to be accurately combined relative to each other. In some such embodiments, the spatial information can include, for example, distance, angle, and rotation of the cannula assemblies relative to one another. In some embodiments, the combined image stream can be, for example, a three-dimensional (“3D”) stereoscopic view. Also, in some embodiments, the system and methods can provide additional functionality and advantages, as described for example in Applicant's co-pending U.S. Provisional Patent Application Ser. No. 63/112,398, the disclosure of which is incorporated by reference herein in its entirety.

Reference will now be made in detail to specific embodiments illustrated in the accompanying drawings. In the following detailed description, numerous specific details are set forth to provide a thorough understanding of the disclosure. However, it will be apparent to one of ordinary skill in the art that embodiments may be practiced without these specific details. In other instances, known methods, procedures, components, circuits, and networks have not been described in detail so as not to unnecessarily obscure aspects of the embodiments.

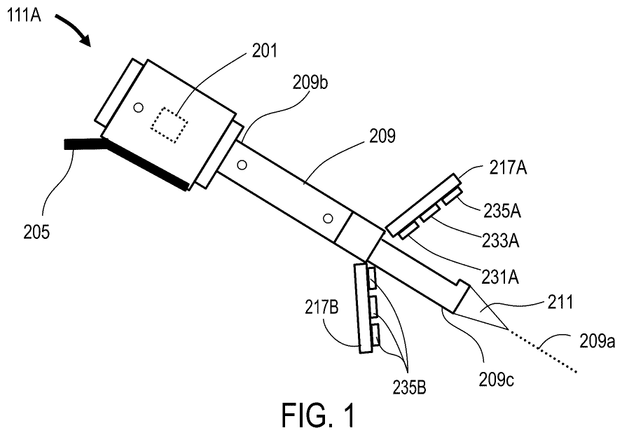

FIG. 1 illustrates one example embodiment. According to this example embodiment, there is provided a cannula assembly 111A. The cannula assembly 111A includes a cannula tube 209 having a longitudinal axis 209a, a proximal end portion 209b, and a distal end portion 209c configured for insertion into a patient. The cannula tube 209 has an internal lumen (not visible in this view) extending from the proximal end portion 209b to the distal end portion 209c.

The cannula tube 209 may be formed of a variety of cross-sectional shapes. For example, the cannula tube 209 can have a generally round or cylindrical, ellipsoidal, triangular, square, rectangular, and D-shaped (in which one side is flat). As mentioned above, in some embodiments, the cannula tube 209 includes an internal lumen 202 into which the obturator 211 is inserted. The obturator 211 can be retractable and/or removable from the cannula tube 209. In some embodiments, the obturator 211 is made of solid, non-transparent material. In another embodiment, all or parts of the obturator 211 are made of optically transparent or transmissive material such that the obturator 211 does not obstruct the view through the camera (discussed below). The obturator 211 may have a tip shape that is configured to penetrate, either via incision or via insertion between tissue planes, through the abdominal wall of the patient.

The cannula assembly 111A also includes first and second sensor housings 217A, 217B coupled to the cannula tube 209 between the proximal and distal ends 209b, 209c of the cannula tube 209 so as to be positioned within the patient when the distal end portion 209c of the cannula tube 209 is inserted into the patient. In this embodiment, the sensor housings 217A, 217B are each movable relative to the cannula tube 209 between a closed position and an open position. The sensor housings 217A, 217B can be integral with the cannula tube 209 or they may be formed as separate components that are coupled to the cannula tube 209. In either case, the sensor housings 217A, 217B can be disposed on or coupled to the cannula tube 209 at positions proximal to the distalmost end of the cannula tube 209 such that they are positioned within the patient's body when the distal end portion of the cannula tube 209 has been inserted into the patient.

In some embodiments, the sensor housings 217A, 217B can be actuated by the actuator handle 205 to open, for example, after being inserted into the patient's body cavity. In some embodiments, the sensor housings 217A, 217B can reside along cannula tube 209 in the distal direction such that they are positioned within the body cavity of a patient (e.g., patient 117) during a surgical procedure. At the same time, sensor housings 217A, 217B can be positioned proximal to the distal end such that they do not interfere with the insertion of the distal end of the cannula tube 209 as it is inserted into a patient. In addition, the sensor housings 217A, 217B can be positioned proximally from distal end to protect the electronic components therein as the distal end is inserted into the patient.

The sensor housings 217A, 217B include light sources 235A, 235B, respectively, and image sensors 231A, 231B, respectively, each configured to provide image data when the sensor housings 217A, 217B are in the open position within the patient. In various embodiments, the light sources 235A, 235B can be dimmable light-emitting devices, such as a LED, a halogen bulb, an incandescent bulb, or other suitable light emitter. Generally, the image sensors 231A, 231B can be devices configured to detect light reflected from the light source 235 and output an image signal. The image sensors 231A, 231B can be, for example, a charged coupled device (“CCD”) or other suitable imaging sensor. In some embodiments, the image sensors 231A, 231B include at least two lenses providing stereo imaging. In some embodiments, the image sensors 231A, 231B can be an omnidirectional camera.

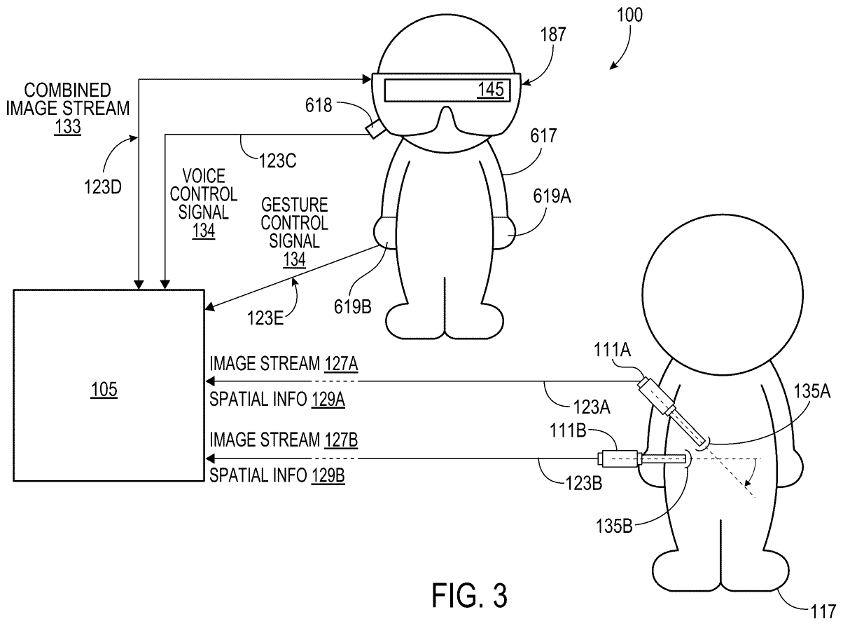

In further embodiments, the cannula assembly 111A may also include a processor or device controller 201 that is configured to receive the image data from the image sensors 231A, 231B and to combine the image data with each other on a separate display device. Additionally or alternatively, the combining of the image data from the image sensors 231A, 231B may take place at least partially, or entirely, in an external processor (such as imaging processor 105 as shown in FIG. 3 and as described in more detail below). Regardless of which processor processes, e.g., combines, the image data from the image sensors 231A, 231B, in various embodiments, the image data are combined with each other by said processor so as to be displayed on an augmented reality device, e.g., an augmented reality headset or pair of augmented reality goggles.

Advantageously, the cannula assembly 111A is configured such that the sensor housings 217A, 217B are rotatable about an axis (not shown in FIG. 1) that is transverse to the longitudinal axis 209a of the cannula tube 209. In this way, when the sensor housings 217A, 217B are in the above-mentioned open position, the sensor housings 217A, 217B are moved to a position that is more lateral relative to the longitudinal axis 209a of the cannula tube 209 as compared to the position of the sensor housings 217A, 217B when in the closed position.

Although not shown herein, it should be understood by those skilled in the art that the cannula assembly 111A (and other cannula assemblies shown and described herein) may include other components and features in addition to those described herein. For example, any of the herein-described cannula assemblies 111A, 111B may include sealing components, such as an instrument seal for sealing around an instrument inserted therethrough, a zero seal for sealing the cannula assembly in the absence of any instrument inserted therethrough, and/or any number of different ports, e.g., insufflation or irrigation ports, for the introduction of various gases or liquids into the surgical site.

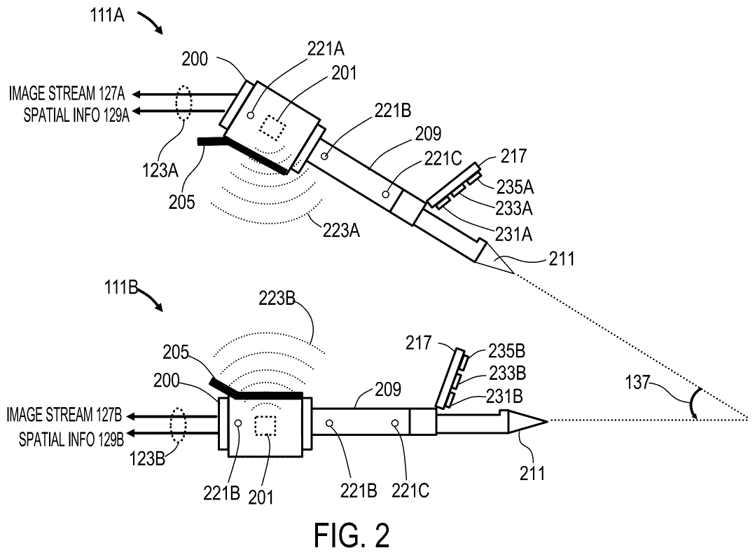

FIG. 2 shows a system illustrating another example embodiment. In this embodiment, there is shown a system in which there are two cannula assemblies 111A, 111B. Although FIG. 2 illustrates two such cannula assemblies, it should be understood that certain advantages may be obtained with a single such cannula assembly, as shown for example in FIG. 1, or that embodiments having more than two cannula assemblies are also contemplated. This embodiment having two cannula assemblies will have additional advantages as shown and described below.

In the embodiment shown in FIG. 2, each of the cannula assemblies 111A, 111B also include, like that shown in FIG. 1, a housing 200, a device controller 201, an actuator handle 205, a cannula tube 209, an obturator 211, and sensor housings 217A, 217B, respectively. In the embodiment shown, each of the cannula assemblies 111A, 111B may also include spatial data components, in this case antennas 221A, 221B, 221C. The cannula tube 209, the obturator 211, and the sensor housings 217A, 217B of the individual cannula assemblies 111A, 111B can be inserted into the body of a patient (e.g., patient 117) and positioned relative to each other, e.g., such as at an angle 137 with respect to each other, so as to provide differing fields-of-view from the sensor housings 217A, 217B.

The device controllers 201, as referenced above, can be one or more devices that process signals and data, e.g., image signals and data. In the embodiment shown in FIG. 2, the device controllers 201 are devices that are configured to generate respective image streams 127A, 127B (see also FIG. 3). Additionally or alternatively, in this embodiment, the device controllers 201 are also configured to generate and/or process spatial information 129A, 129B (see also FIG. 3) of the cannula assemblies 111A, 111B. In some embodiments, the device controller 201 can determine the spatial information 129A, 129B by processing data from spatial sensors (e.g., accelerometers) to determine the relative position, angle, and rotation of the cannula assemblies 111A, 111B.

In some embodiments, the device controller 201 can also determine the spatial information 129A, 129B by processing range information received from sensors such as LiDAR devices 233A, 233B in the sensor housings 217A, 217B, respectively. The LiDAR devices 233A, 233B can include one or more devices that illuminate a region with light beams, such as lasers, and determine distance by measuring reflected light with a photosensor. The distance can be determined based a time difference between the transmission of the beam and detection of backscattered light. For example, using the LiDAR devices 233A, 233B, the device controller 201 can determine spatial information 129A, 129B by sensing the relative distance and rotation of the cannulas 209 or the sensor housings 217A, 217B inside a body cavity.

Additionally or alternatively, in some embodiments, the device controller 201 can process the spatial information 129A, 129B by processing signals received via the antennas 221A, 221B, 221C to determine relative distances of the cannula assemblies 111A, 111B. In various embodiments, the antennas 221A, 221B, 221C can be disposed along the long axis of the cannula assemblies 111A, 111B, e.g., the antennas 221A, 221B, 221C can be placed in a substantially straight line on one or more sides of the cannula assemblies 111A, 111B. For example, two or more lines of the antennas 221A, 221B, 221C can be located on opposing sides of the housing 203 and the cannula tube 209. Although FIG. 2 shows a single line of the antennas 221A, 221B, 221C on one side of the cannula assemblies 111A, 111B, it is understood that the additional lines of the antennas 221A, 221B, 221C can be placed in opposing halves, thirds, or quadrants of the cannula assemblies 111A, 111B.

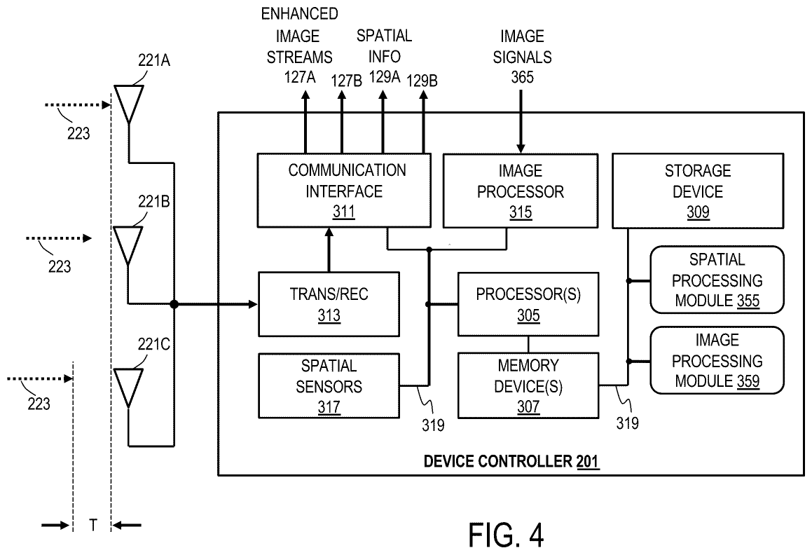

As illustrated in FIG. 2, in some embodiments, the device controllers 201 can additionally or alternatively transmit a ranging signal 223. In some embodiments, the location signals are ultra-wideband (“UWB”) radio signal usable to determine a distance between the cannula assemblies 111A, 111B less than or equal to 1 centimeter based on signal phase and amplitude of the radio signals, as described in IEEE 802.15.4Z. The device controllers 201 can determine the distances between the cannula assemblies 111A, 111B based on the different arrival times of the ranging signals 223A and 223B at their respective antennas 221A, 221B, 221C. For example, referring to FIG. 4, the ranging signal 223A emitted by cannula assembly 111A can be received by cannula assembly 111B at antenna 221C and an amount of time (T) after arriving at antenna 221B. By making a comparison of the varying times of arrival of the ranging signal 223A at two or more of the antennas 221A, 221B, 221C, the device controllers 201 of cannula assembly 111B can determine its distance and angle from cannula assembly 111A. It is understood that the transmitters can be placed at various suitable locations within the cannula assemblies 111A, 111B. For example, in some embodiment, the transmitters can be located in the cannula tubes 209 or in the sensor housings 217A, 217B.

It is understood that, in some embodiments, less than all, e.g., only one or none, of the cannula assemblies 111A, 111B provides spatial information 129. The spatial information, as shown and described herein, is advantageous to ensure that image streams are accurately combined relative to each other; however, it is recognized that other technology may be employed to ensure such accuracy of image combinations.

FIG. 3 shows a block diagram illustrating an example of an environment 100 for implementing the systems and methods described herein. In the embodiment shown in FIG. 3, the environment 100 may include an imaging controller 105. The environment 100 may also include an augmented reality display device 107 worn by a user 617 (e.g., a surgeon) and having a display 145. The environment 100 may further include, in this embodiment, two cannula assemblies 111A, 111B, the distal ends of which, as shown, may be insertable into a surgical site within a patient 117.

The imaging controller 105 can be a computing device connected to the augmented reality display device 107 and the cannula assemblies 111A, 111B through one or more wired or wireless communication channels 123A, 123B, 123D. The communication channels 123A, 123B, 123D may use various serial, parallel, video transmission protocols suitable for their respective signals such as image streams 127A, 127B, combined image stream 133, and data signals, such as spatial information 129A, 129B.

The imaging controller 105 can include hardware, software, or a combination thereof for performing operations. The operations can include receiving the image streams 127A, 127B and the spatial information 129A, 129B from the cannula assemblies 111A, 111B. The operations can also include processing the spatial information 129A, 129B to determine relative positions, angles, and rotations of the cannula assemblies 111A, 111B. In some embodiments, the image streams 127A, 127B and the spatial information 129A, 129B can be substantially synchronous, real-time information captured by the cannula assemblies 111A, 111B. In some embodiments, determining the relative positions, angles, and rotations includes determining respective fields-of-view of the cannula assemblies 111A, 111B. For example, the relative visual perspective can include a relative distance, angle and rotation of the cannula assemblies' 111A, 111B fields-of-view.

The operations of the imaging controller 105 can also include combining the image streams 127A, 127B into the combined image stream 133 based on the spatial information 129A, 129B. In some embodiments, combining the image streams 127A, 127B includes registering and overlaying the images in the fields-of-view of the cannula assemblies 111A, 111B based on the spatial information 129A, 129B. The combined image stream 133 can provide the first image stream 127A as an overlay of the second image stream 127B (or vice versa) so as to provide a user, e.g., a surgeon viewing the display 145 via the augmented reality display device, with enhanced visualization of the patient's surgical space. In some embodiments, the display 145 may be an enhanced stereoscopic 3D view from the perspective of one of the cannula assemblies 111A, 111B, as will be described in additional detail below.

The augmented reality display device 107 can be one or more devices that provide display 145 for a user 617 of the cannula assemblies 111A, 111B. As described above, the augmented reality display device 107 can receive the combined image stream 133 and display it as stereoscopic/3D display 145. The augmented reality display device 107 can be, in some embodiments, a stereoscopic, virtual reality head-mounted display, such as a virtual reality headset and/or goggles.

Having an augmented reality display device 107 in the form of a virtual reality headset can provide various advantages to a user. For example, a virtual reality headset may, in some embodiments, completely cover the visual field of the user, thereby ensuring that the user is not distracted by any visual stimuli in the operating room and can instead focus entirely on the display 145. In addition, having an augmented reality display device 107 in the form of a virtual reality headset can provide additional comfort to the user. Surgical procedures can often take long periods of time, e.g., many hours, and common complaints of many operating room personnel, e.g., particularly surgeons, include the discomfort of being bent over a patient while manipulating the surgical instruments during surgery, while simultaneously needing to crane his or her neck to see a display screen across the room, etc. A virtual reality headset, because it is mounted on the user's head, provides the display directly in the surgeon's line of site, regardless of where or how his or her body is positioned, thereby allowing the surgeon to position his or her body in the most comfortable way for that particular surgeon.

The augmented reality display device 107 may also include additional features that enable additional types of augmented reality functionality. For example, in various embodiments, the augmented reality display device 107 may also include microphone 618. The microphone 618 may be any device that, e.g., is mounted or connected to the augmented reality display device 107 and that the user can speak into during the course of the surgical procedure. The imaging controller 105 may be connected to the microphone 618 of the augmented reality display device 107 through a wired or wireless communication channel 123C. The communication channels 123C may use any serial or parallel audio transmission protocol suitable for transmitting a respective signal, such as voice control signal 134.

The hardware or software (or combination thereof) of the imaging controller 105 can operate to receive the voice control signals 134 and to process the voice control signals 134 to modify some attribute of the display 145. For example, in response to the user speaking an audible instruction into the microphone 618, e.g., an audible instruction to “zoom in” or “zoom out”, an audible instruction to “pan left” or “pan right”, an audible instruction to increase or decrease brightness, etc., the microphone 618 may generate a voice control signal 134 corresponding thereto, and may transmit that corresponding voice control signal 134 to the imaging controller 105. The image controller 105 may then employ its hardware/software to process the voice control signal 134 and to modify the display 145 in accordance with the corresponding voice control signal 134. In this way, the user may optimize the surgical procedure by speaking audible instructions that provide the user with the view of the surgical site that is most helpful to the user. Of course, it should be recognized by any person of skill in the art that there is no limit to the number of different attributes that can be changed by the user, and thus there is no limit to the number of different audible instructions that the image controller 105 can be programmed to process.

The system 100 may also include other features that enable additional types of augmented reality functionality. For example, in various embodiments, the system 100 may also include gesture control input devices 619A, 619B. The gesture control input devices 619A, 619B may be any device that is mounted, connected or worn by the user and via which the user can provide movement. For example, gesture control input devices 619A, 619B may be gloves (or any device that is configured to worn on the hands of the user, e.g., fingertip sensors etc) that are configured to sense the movement of the user's hands or fingers during the course of the surgical procedure. The imaging controller 105 may be connected to the gesture control input devices 619A, 619B through a wired or wireless communication channel 123E. The communication channel 123E may use any serial or parallel transmission protocol suitable for transmitting a respective signal, such as gesture control signal 135.

The hardware or software (or combination thereof) of the imaging controller 105 can operate to receive the gesture control signals 135 and to process the gesture control signals 135 to modify some attribute of the display 145. For example, in response to the user providing, e.g., a pinching-type gesture via the gesture control input devices 619A, 619B, the gesture control devices 619A, 619B may generate a gesture control signal 135 that corresponds to the display 145 being zoomed in. Likewise, in response to the user providing, e.g., a finger-spreading type gesture via the gesture control input devices 619A, 619B, the gesture control devices 619A, 619B may generate a gesture control signal 135 that corresponds to the display 145 being zoomed out. Additionally or alternatively, in response to the user making a sweeping gesture to the left via the gesture control input devices 619A, 619B, the gesture control devices 619A, 619B may generate a gesture control signal 135 that corresponds to the display 145 being panned left, while in response to the user making a sweeping gesture to the right via the gesture control input devices 619A, 619B, the gesture control devices 619A, 619B may generate a gesture control signal 135 that corresponds to the display 145 being panned right. Regardless of which gesture is made via the gesture control input devices, the image controller 105 may then employ its hardware/software to process the gesture control signal 135 and to modify the display 145 in accordance with the corresponding gesture control signal 135. In this way, the user may optimize the surgical procedure by providing gesture-related instructions that provide the user with the view of the surgical site that is most helpful to the user. Of course, it should be recognized by any person of skill in the art that there is no limit to the number of different attributes that can be changed by the user, and thus there is no limit to the number of different to gesture control instructions that the image controller 105 can be programmed to process.

Still further, having these types of features that enable additional types of augmented reality functionality, e.g., having a microphone 618 that enables the display 145 to be selectively modified by a user via voice control signals 134 and/or having gesture control input devices 169A, 169B that enable the display 145 to be selectively modified by a user via gesture control signals 134, can provide still additional advantages to a user. For example, a surgeon typically performs a laparoscopic procedure using multiple cannulas inserted through individual incisions, wherein at least one such cannula and incision is occupied by an illumination/imaging device, such as a traditional endoscope. Typically an operating room technician (someone in the operating room other than the surgeon him or herself) holds the traditional endoscope in place during surgery, and that same person may also assist during the surgery by modifying the display, e.g., such as by moving the endoscope or by changing settings on the endoscope and/or the display device, in accordance with verbal instructions received from the surgeon. Without such another operating room technician being present in the operating room to modify the display in accordance with verbal instructions received from the surgeon, the surgeon would need to hold the endoscope and/or change the settings on the endoscope or display device him or herself, which would greatly hamper the surgeon's ability to conduct the surgery, e.g., by forcing him or her to take his or her hands off of other instruments being used during the surgery, by dividing the surgeon's attention away from the surgical tasks at hand, etc.

In contrast, the system 100 described hereinabove, which may provide the imaging devices 231A, 231B mounted or coupled to the cannula tube 209 itself, may eliminate the need for another operating room technician to hold a separate traditional endoscope. Furthermore, having a microphone 618 that enables the display 145 to be selectively modified by a user via voice control signals 134 and/or having gesture control input devices 169A, 169B that enable the display 145 to be selectively modified by a user via gesture control signals 134, can also potentially eliminate or reduce the need for another operating room technician to be present during a surgery, since the surgeon can use the voice control signals 134 and/or the gesture control signals 135 to modify the display 145 him or herself, without needing to provide verbal instructions to another person to do so.

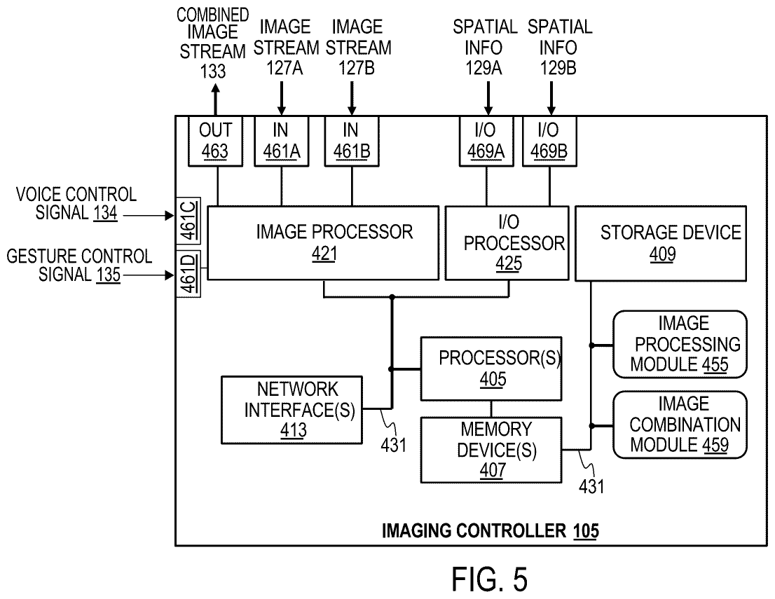

It should be noted that, while the descriptions herein describes various components, operations and functions as potentially being present and/or performed by one or more of the device controller 201 and the imaging controller 105, it is contemplated that the herein-described components, operations and functions may be present or performed entirely in a single one of these devices, that additional controllers/processor may be present that perform any one or more or portions of said operations or functions, and/or that the components described herein may be shared across these devices (and/or such additional processors) such that the device controller 201 and/or the image/navigation controller 105 share responsibility for performing any one or more of the herein-described operations or functions. As will be shown below, FIGS. 4 and 5 illustrate example embodiments in which device controller 201 has components for, and performs, certain operations and functions, while the imaging controller 105 has components for, and performs, certain operations and functions. It should be recognized by those skilled in the art that, in accordance with other embodiments thereof, there may be included processors, either internal or external to the cannula assemblies 111A, 111B, for performing these operations and functions, and that, although described in connection with a certain processor, there is no intent herein to be limited to any particular structure or location of such components, operations or functions. The example embodiment described hereinbelow is merely one way that such processors may be employed.

For example, FIG. 4 shows a functional block diagram illustrating one such example of a device controller 201 in accordance with various aspects described herein. In the embodiment shown, the device controller 201 can include a processor 305, a memory device 307, a storage device 309, a communication interface 311, a transmitter/receiver 313, an image processor 315, spatial sensors 317, and a data bus 319.

In some embodiments, the processor 305 can include one or more microprocessors, microchips, or application-specific integrated circuits. The memory device 307 can include one or more types of random-access memory (RAM), read-only memory (ROM) and cache memory employed during execution of program instructions. The processor 305 can use the data buses 319 to communicate with the memory device 307, the storage device 309, the communication interface 311, the image processor 315, and the spatial sensors 317. The storage device 309 can comprise a computer-readable, non-volatile hardware storage device that stores information and program instructions. For example, the storage device 309 can be one or more, flash drives and/or hard disk drives. The transmitter/receiver 313 can be one or more devices that encodes/decodes data into wireless signals, such as the ranging signal 223.

The processor 305 executes program instructions (e.g., an operating system and/or application programs), which can be stored in the memory device 307 and/or the storage device 309. The processor 305 can also execute program instructions of a spatial processing module 355 and an image processing module 359. The spatial processing module 335 can include program instructions that determine the spatial information 129A, 129B by combining spatial data provided from the transmitter/receiver 313 and the spatial sensors 317. The image processing module 359 can include program instructions that, using the image signals 365 from image sensor 231A, 231B, register and overlay the images to generate the image streams 127A, 127B. The image processor 315 can be a device configured to receive an image signal 365 from an image sensor (e.g., image sensors 231A, 231B) and condition images included in the image signals 365. In accordance with aspects, conditioning the image signals 365 can include normalizing the size, exposure, and brightness of the images. Also, conditioning the image signals 365 can include removing visual artifacts and stabilizing the images to reduce blurring due to motion. Additionally, the image processing module 359 can identify and characterize structures in the images.

In some embodiments, the spatial sensors 317 can include one or more of, piezoelectric sensors, mechanical sensors (e.g., a microelectronic mechanical system (“MEMS”), or other suitable sensors for detecting the location, velocity, acceleration, and rotation of the cannula assemblies (e.g., cannula assemblies 111A, 111B).

It is noted that the device controller 201 is only representative of various possible equivalent-computing devices that can perform the processes and functions described herein. To this extent, in some embodiments, the functionality provided by the device controller 201 can be any combination of general and/or specific purpose hardware and/or program instructions. In each embodiment, the program instructions and hardware can be created using standard programming and engineering techniques.

Likewise, FIG. 5 shows a functional block diagram illustrating an imaging controller 105 in accordance with one such example embodiment. The imaging controller 105 can include a processor 405, a memory device 407, a storage device 409, a network interface 413, an image processor 421, an I/O processor 425, and a data bus 431. Also, the imaging controller 105 may include input connections 461A, 461B for receiving image data streams 127A, 127B, respectively. Further, the imaging controller 105 may include input/output connections 469A, 469B that receive/transmit spatial data signals 129A, 129B to and from I/O processor 425. Also, the image controller 105 may include input connections 461C, 461D for receiving voice control signals 134 and gesture control signals 135, respectively. Still further, the image controller 105 may include output connection 463 that transmits the combined image stream 133 from the image processor 421 to, e.g., the augmented reality display device 107.

In embodiments, the imaging controller 105 can include one or more microprocessors, microchips, or application-specific integrated circuits. The memory device 407 can include one or more types of random-access memory (RAM), read-only memory (ROM) and cache memory employed during execution of program instructions. Additionally, the imaging controller 105 can include one or more data buses 431 by which it communicates with the memory device 407, the storage device 409, the network interface 413, the image processor 421, and the I/O processor 425. The storage device 409 can comprise a computer-readable, non-volatile hardware storage device that stores information and program instructions. For example, the storage device 409 can be one or more, flash drives and/or hard disk drives.

The I/O processor 425 can be connected the processor 405 and can include any device that enables an individual to interact with the processor 405 (e.g., a user interface) and/or any device that enables the processor 405 to communicate with one or more other computing devices using any type of communications link. The I/O processor 425 can generate and receive, for example, digital and analog inputs/outputs according to various data transmission protocols.

The processor 405 executes program instructions (e.g., an operating system and/or application programs), which can be stored in the memory device 407 and/or the storage device 409. The processor 405 can also execute program instructions of an image processing module 455 and an image combination module 459. The image processing module 455 can be configured to stabilize the images to reduce the blurring, compensate for differences in tilt and rotation, remove reflections and other visual artifacts from the images, and normalize the images. Additionally, the image processing module 455 can be configured to identify and characterize structures, such as tools or tissues, in the images. Further, the imaging processing module 455 can be configured to determine obstructions in the overlapping fields of view and process the images streams 127A, 127B to remove the obstructions.

The image combination module 459 can be configured to analyze images received in image streams 127A, 127B from the cannula assemblies 111A, 111B and combine them into a single image stream 133 based, e.g., on the spatial information 129A, 129B. In some embodiments, the image combination module 459 generates the combined image stream 133 by registering and overlaying the image streams 127A,127B based on the respective fields-of-view of the cannula assemblies. In some embodiments, either of the cannula assemblies 111A, 111B can be selected by a user, e.g., via I/O processor 425, as a primary cannula assembly, and the image combination module 459 can generate the combined image stream 133 by using the image stream of the secondary cannula assembly to augment the primary image stream. The combined image stream 133 can also provide a stereoscopic 3D view from the perspective of the primary cannula assembly. In some embodiments, the combined image stream 133 lacks the obstructions removed by the image processing module 455. In some embodiments, the combined image stream 133 may also include image data provided by a secondary imaging system, e.g., an imaging system that provides alternate image data (not shown).

The image processor 421 may also be configured, as set forth above, to process the additional augmented reality functionality. For example, in embodiments in which the augmented reality display device 107 also includes the microphone 618, the imaging controller 105 may be connected to the microphone 618 such that the imaging controller 105 receives, via input connection 461C, the voice control signals 134 and processes the voice control signals 134 to modify some attribute of the display 145, e.g., to “zoom in” or “zoom out”, to “pan left” or “pan right”, to increase or decrease brightness, etc. The image controller 105 may then employ its hardware/software to process the voice control signal 134 and to modify the combined image stream 133 that is transmitted to the display 145 in accordance with the corresponding voice control signals 134. Likewise, in embodiments in which the system 100 also includes gesture control input devices 619A, 619B, the imaging controller 105 may be connected to the gesture control input devices 619A, 619B such that the imaging controller 105 receives, via input connection 461D, the gesture control signals 135 and processes the gesture control signals 135 to modify some attribute of the display 145. The image controller 105 may then employ its hardware/software to process the gesture control signal 135 and to modify the combined image stream 133 that is transmitted to the display 145 in accordance with the corresponding gesture control signals 135. Of course, and as mentioned previously, it should be recognized by any person of skill in the art that there is no limit to the number of different attributes that can be changed by the user via the voice control signals 134 and/or the gesture control signals 135, and thus there is no limit to the number of different voice and/or gesture control instructions that the image controller 105 can be programmed to process.

It is noted that the imaging controller 105 is only representative of various possible equivalent-computing devices that can perform the processes and functions described herein. To this extent, in some embodiments, the functionality provided by the imaging controller 105 can be any combination of general and/or specific purpose hardware and/or program instructions. In each embodiment, the program instructions and hardware can be created using standard programming and engineering techniques.

The particular embodiments described in this application are not limiting, as they are intended as illustrations of various aspects. Many modifications and variations can be made without departing from its spirit and scope, as will be apparent to those skilled in the art. Functionally equivalent methods and apparatuses, in addition to those enumerated herein, will be apparent to those skilled in the art from the foregoing descriptions. Such modifications and variations are intended to fall within the scope of the appended claims. Only the terms of the appended claims are intended to be limiting, along with the full scope of equivalents to which such claims are entitled. It is also to be understood that the terminology used herein, e.g., “and”, “or”, “including”, “at least” as well as the use of plural or singular forms, etc., is for the purpose of describing examples of embodiments and is not intended to be limiting.

1. A surgical system for performing a surgical procedure on a patient, comprising:
a cannula assembly including a cannula tube having a longitudinal axis, a proximal end portion and a distal end portion configured for insertion into a patient, the cannula tube having a housing coupled to the cannula tube between the proximal and distal ends of the cannula tube so as to be positioned within the patient when the distal end of the cannula tube is inserted into the patient, the housing movable relative to the cannula tube between a closed position and an open position, the housing including an image sensor configured to provide image data of the patient's anatomy when the housing is in the open position within the patient; and
an augmented reality display device configured to receive the image data and to display the image data to a user.

2. The surgical system of claim 1, wherein the augmented reality display device includes a pair of augmented reality goggles configured to be worn by the user.

3. The surgical system of claim 1, wherein the system further comprises:
an augmented reality processor configured to process the image data for display to the user via the augmented reality display device.

4. The surgical system of claim 3, further comprising:
a microphone,
wherein the augmented reality processor is configured to receive a voice control signal from the microphone and to process the voice control signal so as to control at least one attribute of the image displayed to the user via the augmented reality display device.

5. The surgical system of claim 4, wherein the microphone is mounted on the augmented reality display device.

6. The surgical system of claim 3, further comprising:
a gesture control input device,
wherein the augmented reality processor is configured to receive a gesture control signal from the gesture control input device and to process the gesture control signal to control at least one attribute of the image displayed to the user via the augmented reality display device.

7. The surgical system of claim 6, wherein the gesture control input device includes one of a glove or a fingertip sensor.

8. The surgical system of claim 1, further comprising:
a second imaging device configured for insertion into a patient and to provide second image data of the patient's anatomy; and
an image processor configured to receive the first and second image data and to process the first and second image data so as to generate a combined image displayed to the user via the augmented reality display device. ;
wherein the cannula assembly includes spatial data components that provide spatial data related to the position of the cannula assembly, the image processor configured to generate the combined image based at least in part on the spatial data.

9. The surgical system of claim 8, wherein the cannula assembly includes a second housing coupled to the cannula tube between the proximal and distal ends of the cannula tube so as to be positioned within the patient when the distal end of the cannula tube is inserted into the patient, the second housing movable relative to the cannula tube between a closed position and an open position,
wherein the second imaging device is an image sensor mounted in the second housing.

10. (canceled)

11. A surgical system for performing a surgical procedure on a patient, comprising:
a cannula assembly including a cannula tube having a longitudinal axis, a proximal end portion and a distal end portion configured for insertion into a patient, the cannula tube having a housing coupled to the cannula tube between the proximal and distal ends of the cannula tube so as to be positioned within the patient when the distal end of the cannula tube is inserted into the patient, the housing movable relative to the cannula tube between a closed position and an open position, the housing including an image sensor configured to provide image data of the patient's anatomy when the housing is in the open position within the patient;
a processor; and
a microphone;
wherein the processor is configured to receive a voice control signal from the microphone and to process the voice control signal so as to control at least one attribute of an image displayed to a user on a display device.

12. The surgical system of claim 11, wherein the processor is an augmented reality processor configured to process the image data.

13. The surgical system of claim 12, further comprising:
a pair of augmented reality display goggles configured to be worn by the user and to display the image data to the user;
a wherein the microphone is mounted on the augmented reality display goggles,
wherein the augmented reality processor is configured to receive a voice control signal from the microphone and to process the voice control signal so as to control at least one attribute of the image displayed to the user via the augmented reality display goggles.

14. The surgical system of claim 2, wherein the display device is an augmented reality display device and the microphone is mounted on the augmented reality display device.

15. The surgical system of claim 12, further comprising:
a gesture control input device,
wherein the augmented reality processor is configured to receive a gesture control signal from the gesture control input device and to process the gesture control signal to control at least one attribute of the image displayed to the user via the augmented reality display goggles.

16. The surgical system of claim 15, wherein the gesture control input device includes one of a glove or a fingertip sensor.

17. The surgical system of claim 11, further comprising:
a second imaging device configured for insertion into a patient and to provide second image data of the patient's anatomy; and
an image processor configured to receive the first and second image data and to process the first and second image data so as to generate a combined image displayed to the user via the augmented reality display goggles;
wherein the cannula assembly includes spatial data components that provide spatial data related to the position of the cannula assembly, the image processor configured to generate the combined image based at least in part on the spatial data.

18. The surgical system of claim 17, wherein the cannula assembly includes a second housing coupled to the cannula tube between the proximal and distal ends of the cannula tube so as to be positioned within the patient when the distal end of the cannula tube is inserted into the patient, the second housing movable relative to the cannula tube between a closed position and an open position,
wherein the second imaging device is an image sensor mounted in the second housing.

19-23. (canceled)

24. The surgical system of claim 11, wherein the voice control signal provides an audible instruction of one or both of zoom in/out and pan left/right.

25. The surgical system of claim 11, wherein the voice control signal provides an audible instruction to increase or decrease brightness of the image displayed on the display device.

26. The surgical system of claim 4, wherein the voice control signal provides an audible instruction of one or both of zoom and brightness of the image displayed on the display device.
